(19) United States
(12) Patent Application Publication
(10) Pub. No.: US 2025/0364129 A1
(43) Pub. Date: Nov. 27, 2025

(54) WIRE USAGE MONITORING

(71) Applicant: KONINKLIJKE PHILIPS N.V., EINDHOVEN (NL)

(72) Inventors: Marian Karin Joke Dekker, Eindhoven (NL); Otto Paulus Henrikus Kokke, Eindhoven (NL)

(21) Appl. No.: 18/867,176

(22) PCT Filed: May 4, 2023
(86) PCT No.: PCT/EP2023/061738

§ 371 Date: Nov. 19, 2024

Related U.S. Application Data
(60) Provisional application No. 63/343,626, filed on May 19, 2022.

(51) Int. Cl. G16H 40/63; A61B 5/00; G16H 40/67

(52) U.S. Cl. CPC G16H 40/63; A61B 5/002; G16H 40/67; A61B 2562/226

(57) ABSTRACT

A wire system (100A) includes a wire body (110) and a controller (150). Data are communicated by the wire body (110). The controller (150) is dedicated to the wire body (110) and includes a memory (151) that stores instructions and a processor (1010) that executes the instructions. When executed by the processor (1010), the instructions cause the controller (150) to detect usage of the wire system (100A) when data are communicated; and update status information for the wire system (100A) stored in the memory (151) to track usage of the wire system (100A).

### BACKGROUND

Cables are often lost, whether at hospital departments or in other contexts. Additionally, the intended use for some types of specialty cables is often not particularly readily ascertainable to individuals, such as when the specialty cables are placed in temporary storage with other types of cables. For at least some contexts such as hospitals, no mechanism exists to logically check dedicated memories tied to such cables to determine purpose of the cables. Additionally, in some contexts cables may deteriorate over time, and this may degrade the reliability of data communicated over the cables. The lack of a mechanism to logically check such dedicated memories tied to such tables also results in an inability to logically check for reasons that might explain the degradation such as age.

### SUMMARY

According to an aspect of the present disclosure, a wire system includes a wire body and a controller. Data are communicated by the wire body. The controller is dedicated to the wire body and includes a memory that stores instructions and a processor that executes the instructions. When executed by the processor, the instructions cause the controller to detect usage of the wire system when data are communicated; and update status information for the wire system stored in the memory to track usage of the wire system.

According to another aspect of the present disclosure, an operational method of a wire system includes detecting usage of the wire system when data are communicated; and updating status information for the wire system stored in a memory of the wire system to track usage of the wire system.

According to another aspect of the present disclosure, a tangible non-transitory computer readable storage medium that stores a computer program. The computer program, when executed by a processor, causes a controller to detect usage of the wire system when data are communicated; and update status information for the wire system stored in a memory of the wire system to track usage of the wire system.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for wire usage monitoring, in accordance with a representative embodiment.

FIG. 1B illustrates a system for wire usage monitoring, in accordance with a representative embodiment.

FIG. 2A illustrates another system for wire usage monitoring, in accordance with a representative embodiment.

FIG. 2B illustrates another system for wire usage monitoring, in accordance with a representative embodiment.

FIG. 3A illustrates another system for wire usage monitoring, in accordance with a representative embodiment.

FIG. 3B illustrates another system for wire usage monitoring, in accordance with a representative embodiment.

FIG. 4 illustrates a method for wire usage monitoring, in accordance with a representative embodiment.

FIG. 5 illustrates another method for wire usage monitoring, in accordance with a representative embodiment.

FIG. 6 illustrates a user interface for wire usage monitoring, in accordance with a representative embodiment.

FIG. 7 illustrates another method for wire usage monitoring, in accordance with a representative embodiment.

FIG. 8 illustrates a controller for wire usage monitoring, in accordance with a representative embodiment.

FIG. 9 illustrates another system for wire usage monitoring, in accordance with a representative embodiment.

FIG. 10 illustrates a computer system, on which a method for wire usage monitoring is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a,’ ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims.

As described herein, data passed through wires may be checked by elements dedicated to the wires, such as controllers comprising a processor that processes instructions, and a memory that stores the instructions, at least some of the data, and historical information related to the data. Additionally, as described herein a wire system may provide functionality that enhances abilities of electronic devices, such as abilities to interface with sensors which cannot directly interface with the electronic devices and abilities to interpret data which may be uninterpretable by the electronic devices.

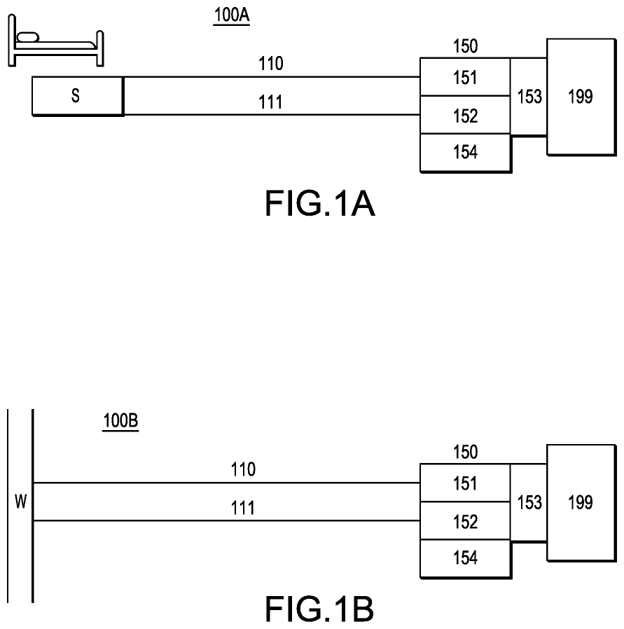

FIG. 1A illustrates a system for wire usage monitoring, in accordance with a representative embodiment.

The wire system 100A includes a wire body 110 and a controller 150. The wire body 110 includes a wire portion 111. The controller 150 includes at least a memory 151 that stores instructions, a processor 152 that executes the instructions, an interface 153 and an interface 154. For example, the interface 153 may be a first interface and the interface 154 may be a second interface, though the controller 150 may include more than two interfaces.

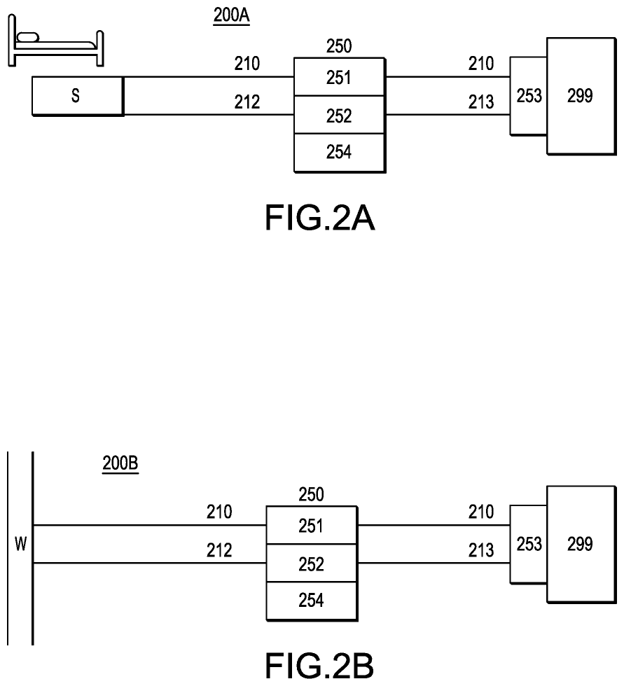

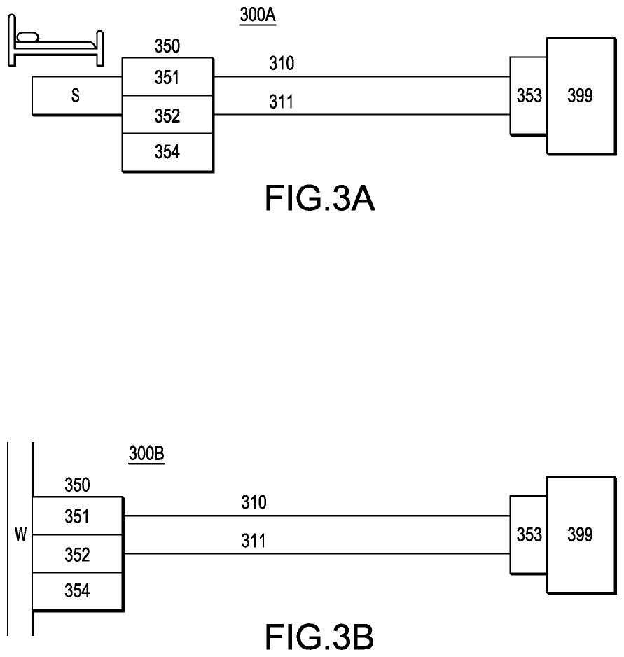

The wire body 110 may include multiple layers or elements such as an outer sheath (not shown) and the wire portion 111. The wire portion 111 may comprise copper, fiber or another known type of medium for communicating data via wire systems. In FIG. 1A, the wire body 110 is connected to a sensor S at a patient bed, though the sensor S is representative of any sensor which provides information directly or indirectly to an electronic device 199. In FIG. 1A, FIG. 2A and FIG. 3A, the sensor S may be a patient sensor which senses physiological characteristics of patients or other users, such as in a hospital or other medical facility. In FIG. 1A, the electronic device 199 is connected to the wire body 110 through the interface 153 of the controller 150. For example, the interface 153 may be configured to interface the wire system 100A to a medical monitor as the electronic device 199. More particularly, the interface may be configured to interface the controller 150 to a medical monitor as the electronic device 199.

For the controller 150, the memory 151 may comprise random access memory (RAM), such as dynamic RAM (DRAM), and this may be true of other memories in wire systems described herein. The processor 152 may comprise a microprocessor, and this may be true of other memories in wire systems described herein. The memory 151 and the processor 152 may have a relatively small form factor, such as a form factor comparable to the form factor of the wire body 110, and this may be true of other wire systems described herein.

Additionally, the controller 150 may receive and process all data that passes through the wire body 110, so that the controller 150 receives the data, and processes the data. The controller 150 may retransmit the data. Alternatively, the controller 150 may include a splitter (not shown), that splits a feed from the wire body 110 so that the data passes through the controller 150 and also is separately received and processed by the controller 150. In some embodiments, the controller 150 may receive all of the data that passes through the wire body 110, but only process a subset of the data that passes through the wire body 110. For example, the controller 150 may receive a new data stream and only process the first, e.g., 8, 16, 24 or 32, bytes of data such as to check for a type or format of the data and/or whether the data are addressed to the electronic device 199 or the controller 150. When the data are addressed to the controller 150, the controller 150 may selectively process more than the first bytes of data such as all of the data. When the data are not addressed to the controller 150, the controller 150 may selectively top processing the data.

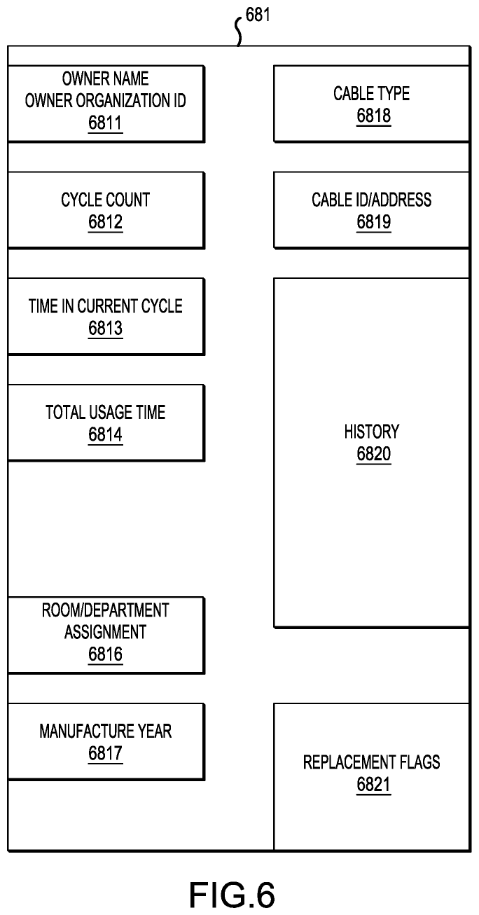

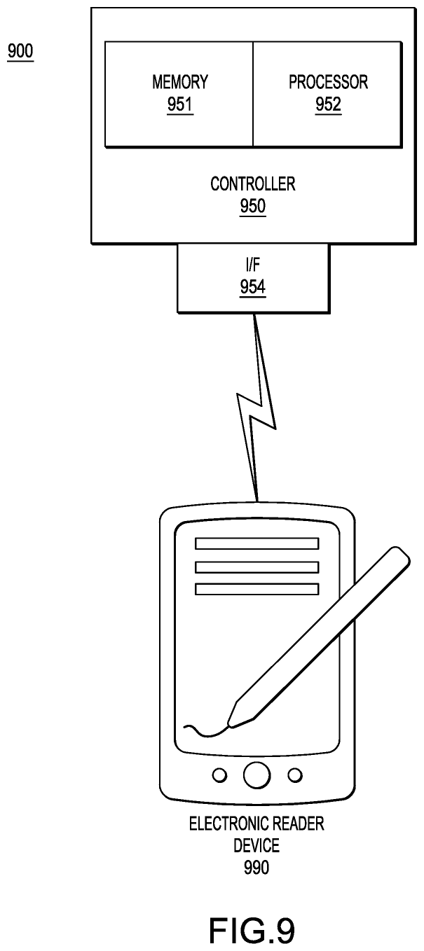

The interface 154 may be an interface used to connect to an external device, either wirelessly or by another wire. For example, the interface 154 may be used to connect the controller 150 to a portable electronic reader device (not shown) via near-field communications (NFC) or WiFi or radio frequency identification (RFID) so that the portable electronic reader device may read data from the memory 151. The interface 154 may alternatively comprise a port such as a serial port or a parallel port used by the portable electronic reader device to read data from the memory 151. An example of a user interface 681 that may be used to read data from the memory 151 is shown in and described with respect to FIG. 6. An example of an electronic reader device is shown in and described with respect to FIG. 9.

Use of a wireless module as the interface 154 may minimize the number of pins required for the controller 150, and may minimize cleaning concerns. A wireless module as the interface 154 may allow an electronic reader device shown in FIG. 9 to retrieve some or all cable data stored in the memory 151.

Examples of the sensor S include pulse oximetry sensors, temperature sensors, airflow sensors, oxygen sensors and pressure sensors. In some embodiments, the sensor S may include other types of sensors, such as sensors used to detect when medical equipment has become separated from patients.

The controller 150 and other controllers described herein add intelligence and data storage to smart cables in order to provide one or more capabilities that do not otherwise exist for cables that only operate as communication mediums. The added capabilities may include an ability to register cable owners and cable details, an ability to track movement of the cables such as connections to electronic medical devices, placement in specific rooms, use by particular users such as nurses, and movement between departments. Added capabilities may also include cable status tracking, such as by using information on the year of manufacture, total number of measurements within a cycle, total number of cycles, total usage time, and more. Additionally, the controller 150 and other controllers described herein may also generate, store and present flags based on monitoring of the data, including flags based on substantive monitoring such as checks showing that the data may be corrupted so that the set up and connection should be checked and confirmed. One or more flags may also specify that the cable needs to be replaced immediately, such as a red flag that is triggered based on age of the cable or deviations between input data and output data. The controller 150 and other controllers described herein may also be used to check compatibility with connected electronic devices, and may indicate whether the cable can be properly used for a particular purpose such as a planned measurement which is outside of the cable's intended use. The controller 150 may also receive information from connected electronic devices, such as information of the room number, department, and user(s), and may provide tracking functions for numbers of measurements, usage time, and more.

In some embodiments, the wire system 100A and other wire systems described herein may provide functionality that would typically be provided by medical monitors such as the electronic device 199. For example, the wire system 100A may be specifically configured to interface with a particular type of sensor S which is otherwise not configured to interface with the electronic device 199. The controller 150 may execute instructions to interpret data received from the sensor S, and provide the data from the sensor S to the electronic device 199. In some embodiments, the controller 150 may generate and provide additional data to the electronic device 199, such as user interface data to provide labels and other context for the data from the sensor S.

FIG. 1B illustrates another system for wire usage monitoring, in accordance with a representative embodiment.

The wire system 100B includes the same elements as the wire system 100A in FIG. 1A, including a wire body 110 and a controller 150. The wire body 110 includes a wire portion 111. The controller 150 includes at least a memory 151 that stores instructions, a processor 152 that executes the instructions, an interface 153 and an interface 154. To the extent possible, descriptions of elements of the wire system 100B which are redundant to descriptions of the wire system 100A are not repeated.

In FIG. 1B, the wire body 110 is connected to a wall W, though the wall W is representative of portions of systems, rooms and buildings through which data connections and/or power connections are provided to electronic equipment such as electronic devices. The wire system 100B is not particularly restricted to patients, medical facilities or a medical context. For example, the wire system 100B may be used to connect an electronic device 199 to power and/or data through the wall W at a home or office. The controller 150 may be configured to monitor usage of the wire system 100B, such as one or more specific type(s) of data that pass across the wire system 100B.

FIG. 2A illustrates another system for wire usage monitoring, in accordance with a representative embodiment.

The wire system 200A includes a wire body 210, a controller 250 and an interface 253. The wire body 210 includes a first wire portion 212 and a second wire portion 213. The controller 250 includes at least a memory 251 that stores instructions, a processor 252 that executes the instructions, and an interface 254.

The wire body 210 is again connected to a sensor S at a patient bed, such as in a medical facility. In FIG. 2A, the electronic device 299 is connected to the wire body 210 through the interface 253. The interface 253 may be provided as an independent element, as an element integrated with the wire body 210, or as an element integrated with the electronic device 299.

The interface 254 may again be an interface used to connect to an external device, either wirelessly or by another wire. For example, the interface 254 may be used to connect the controller 250 to a portable electronic reader device (not shown) via near-filed communications (NFC) or WiFi so that the portable electronic reader device may read data from the memory 251. The interface 254 may alternatively comprise a port such as a serial port or a parallel port used by the portable electronic reader device to read data from the memory 251. An example of a user interface 681 that may be used to read data from the memory 251 is shown in and described with respect to FIG. 6. An example of an electronic reader device is shown in and described with respect to FIG. 9.

Compared to the embodiment of FIG. 1A, in the embodiment of FIG. 2A the controller 250 is provided between the first wire portion 212 and the second wire portion 213.

FIG. 2B illustrates another system for wire usage monitoring, in accordance with a representative embodiment.

In the embodiment of FIG. 2B, the wire system 200B includes the same elements and configuration as the wire system 200A in FIG. 2A, including a wire body 210, a controller 250 and an interface 253. The wire body 210 includes a first wire portion 212 and a second wire portion 213. The controller 250 includes at least a memory 251 that stores instructions, a processor 252 that executes the instructions, and an interface 254. To the extent possible, descriptions of elements of the wire system 200B which are redundant to descriptions of the wire system 100A, the wire system 100B, or the wire system 200A are not repeated.

The wire body 210 in FIG. 2B is connected to a wall W, and the wall W is representative of portions of systems, rooms and buildings through which data connections and power connections are provided to electronic equipment such as electronic devices. The wire system 200B is not particularly restricted to patients, medical facilities or a medical context. For example, the wire system 200B may be used to connect an electronic device 299 to power and/or data through the wall W at a home or office. The controller 250 may be configured to monitor usage of the wire system 200B, such as one or more specific type(s) of data that pass across the wire system 200B.

FIG. 3A illustrates another system for wire usage monitoring, in accordance with a representative embodiment.

The wire system 300A includes a wire body 310, a controller 350 and an interface 353. The wire body 310 includes a wire portion 311. The controller 350 includes at least a memory 351 that stores instructions, a processor 352 that executes the instructions, and an interface 354.

In FIG. 3A, the controller 350 is connected to a sensor S at a patient bed. In FIG. 3A, the electronic device 399 is connected to the wire body 310 through the interface 353. The interface 353 may be provided as an independent element, as an element integrated with the wire body 310, or as an element integrated with the electronic device 399.

The interface 354 may again be an interface used to connect to an external device, either wirelessly or by another wire. For example, the interface 354 may be used to connect the controller 350 to a portable electronic reader device (not shown) via near-filed communications (NFC) or WiFi so that the portable electronic reader device may read data from the memory 351. The interface 354 may alternatively comprise a port such as a serial port or a parallel port used by the portable electronic reader device to read data from the memory 351. An example of a user interface 681 that may be used to read data from the memory 351 is shown in and described with respect to FIG. 6. An example of an electronic reader device is shown in and described with respect to FIG. 9.

Compared to the embodiment of FIG. 1A and the embodiment of FIG. 2A, in the embodiment of FIG. 3A the controller 350 is provided between the wire portion 311 and the sensor S.

FIG. 3B illustrates another system for wire usage monitoring, in accordance with a representative embodiment.

In the embodiment of FIG. 3B, the wire system 300B includes the same elements and configuration as the wire system 300A in FIG. 3A, including a wire body 310, a controller 350 and an interface 353. The wire body 310 includes a wire portion 311. The controller 350 includes at least a memory 351 that stores instructions, a processor 352 that executes the instructions, and an interface 354. To the extent possible, descriptions of elements of the wire system 300B which are redundant to descriptions of the wire system 100A, the wire system 100B, the wire system 200A, the wire system 200B or the wire system 300A are not repeated.

In FIG. 3B, the controller 350 is connected to a wall W, and the wall W is representative of portions of systems, rooms and buildings through which data connections and power connections are provided to electronic equipment such as electronic devices. The wire system 300B is not particularly restricted to patients, medical facilities or a medical context. For example, the wire system 300B may be used to connect an electronic device 399 to power and/or data through the wall W at a home or office. The controller 350 may be configured to monitor usage of the wire system 300B, such as one or more specific type(s) of data that pass across the wire system 300B.

Compared to the embodiment of FIG. 1B and the embodiment of FIG. 2B, in the embodiment of FIG. 3B the controller 350 is provided between the wire portion 311 and the wall W.

In the wire system 100A of FIG. 1A, the wire system 200A of FIG. 2A and the wire system 300A of FIG. 3A, connections between the wire bodies, the controllers and the sensor S may use interfaces of known types. Additionally, in the wire system 100B of FIG. 1B, the wire system 200B of FIG. 2B and the wire system 300B of FIG. 3B, connections between the wire bodies, the controllers and the wall W may use interfaces of known types. For example, connections may be made using adapters, such as male/female pairs. Connections may be made via threaded surfaces or pins. Insofar as the controllers are configured to be fixed, either permanently or detachably, to the wire bodies, the controllers and the wire bodies may be assembled together during manufacture, such as via welding, molding, or another form of permanent or semi-permanent process. Additionally, the controllers are configured to receive data from data connections such as copper wires or fiber, and process the received data either by analyzing some or all of the received data, or by at least passing the received data through to either another portion of the wire body to the sensor S, to the wall W, or to the electronic equipment.

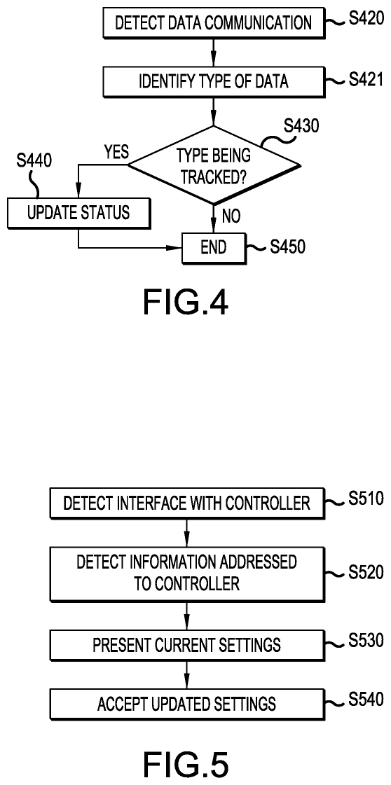

FIG. 4 illustrates a method for wire usage monitoring, in accordance with a representative embodiment.

The method of FIG. 4 starts at S420 by detecting a data communication. For example, the controller 150, the controller 250 or the controller 350 may detect that data are being communicated across the wire body 110, the wire body 210 or the wire body 310 respectively when data are received.

At S421, the method of FIG. 4 includes identifying the type of data. For example, the controller 150, the controller 250 or the controller 350 may be programmed to ignore some types of data and may be programmed to act for other types of data.

At S430, a determination is made whether the type of data identified at S421 is a type of data being tracked. For example, the determination at S430 may include checking a type of format of the data indicated in a header of a packet that carries the data as a payload.

If the data are of a type being tracked (S430=Yes), at S440 the method of FIG. 4 includes updating a status. For example, the controller 150, the controller 250 or the controller 350 may record the time the data are received, the length of time the data are communicated, or another form of indication that data of a type being tracked is being communicated. The updated status may be updated by updating a count of usage time of the wire system 100A or the wire system 100B, the wire system 200A or the wire system 200B or the wire system 300A or the wire system 300B.

If the data are not of a type being tracked (S430=No), the method of FIG. 4 ends at S450.

FIG. 5 illustrates another method for wire usage monitoring, in accordance with a representative embodiment.

At S510, the method of FIG. 5 starts with detecting an interface with a controller. For example, the controller 150, the controller 250 or the controller 350 may detect that an electronic reader device has physically connected or wirelessly connected. The physical connection may be by an electronic reader device plugging into the controller 150, the controller 250 or the controller 350, whereas the wireless connection may be by the electronic reader device initiating a connection request via WiFi or near-field communication with the controller 150, the controller 250 or the controller 350. For example, the interface 154, the interface 254 or the interface 354 may be largely or entirely dedicated to interfacing with electronic reader devices in order to provide access to status information stored in the memory 151, the memory 251 or the memory 351, respectively.

At S520, the method of FIG. 5 includes detecting information addressed to the controller. For example, the controller 150, the controller 250 or the controller 350 may detect an inquiry or request to retrieve information from the electronic reader device. The electronic reader device may specifically provide a unique identification for the wire system 100B, the wire system 200B or the wire system 300B that reflects that the electronic reader device is authorized to retrieve information from the controller 150, the controller 250 or the controller 350, respectively.

At S530, the method of FIG. 5 includes presenting current settings. For example, the controller 150, the controller 250 or the controller 350 may provide settings showing that the wire system 100B, the wire system 200B or the wire system 300B belongs to a particular person or organization, is assigned to a particular room or area, is movable or immovable, or other forms of settings.

At S540, the method of FIG. 5 includes accepting updating settings for the wire system 100B, the wire system 200B or the wire system 300B. Updated settings for the wire system 100B, the wire system 200B or the wire system 300B may be accepted after verification that an electronic reader device is authorized to update the settings, such as by requiring a password, checking an authorization level, and/or by other known mechanisms for verifying authority to perform functions.

The method of FIG. 5 may be performed in order to allow changes is assignments for the wire system 100B, the wire system 200B or the wire system 300B.

FIG. 6 illustrates a user interface for wire usage monitoring, in accordance with a representative embodiment.

The user interface 681 in FIG. 6 may be presented when an authorized electronic reader device interfaces with the controller 150, the controller 250 or the controller 350. The user interface may provide any of the types of information shown in FIG. 6, and other types of information not shown. For example, the user interface may include:
owner name and owner organizational ID 6811.
cycle count 6812, such as count of how many patients the wire system 100B, the wire system 200B or the wire system 300B has been assigned to for use.
time in current cycle 6813, such as a total amount of time since the settings were last updated at S540.
total usage time 6814, such as a count of usage reflected by the total amount of time the wire system 100B, the wire system 200B or the wire system 300B has been assigned to any patient.
room/department assignment 6816, such as a specific hospital room and hospital department to which the wire system 100B, the wire system 200B or the wire system 300B has been assigned.
manufacture year 6817, such as the year the controller 150, the controller 250 or the controller 350 was first activated.
cable type 6818, such as the type of data for which the cable is supposed to be used.
cable ID/address, such as a unique communication address assigned to the cable
history 6820, such as the history of each patient, room, department or clinician the cable has been assigned to.
replacement flags 6821, such as flags which are automatically set once the cable has been used for too long, too many cycled, without being relieved, or flags for any other reason which might indicate that the cable should be replaced.
other types of information (not shown), including an open field that may be left open to information deemed pertinent to a specific context, and/or a field later dedicated to a particular type of information not specified in the fields already shown in the user interface 681.

A portable electronic device used to read data from a controller 150, a controller 250 or a controller 350 may include a display by which the user interface 681 provides information from the memory 151, the memory 251 or the memory 351. The portable electronic device may include a display such as a display that is configured to remotely connect to the controller 150, the controller 250 or the controller 350. The display may be a monitor such as a computer monitor, a display on a mobile device, an augmented reality display, a television, an electronic whiteboard, or another screen configured to display electronic imagery.

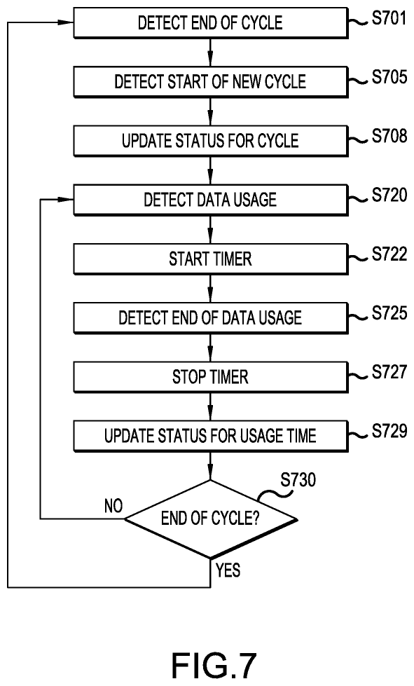

FIG. 7 illustrates another method for wire usage monitoring, in accordance with a representative embodiment.

The method of FIG. 7 starts at S701 with detecting the end of a usage cycle. For example, the controller 150, the controller 250 or the controller 350 may detect that the corresponding system has been disconnected from both a corresponding medical device at one end and from the wall W at the other end.

At S705, the start of a new usage cycle is detected. For example, the controller 150, the controller 250 or the controller 350 may detect that the corresponding wire system 100A or wire system 100B, wire system 200A or wire system 200B or wire system 300A or wire system 300B has been connected to both a corresponding medical device at one end and to the wall W at the other end.

At S708, the status of a system is updated to account for the new cycle which is detected at S705. For example, a register at a dedicated memory address in the memory 151, the memory 251 or the memory 351 may be incremented by 1.

At S720, data usage is detected. For example, the controller 150, the controller 250 or the controller 350 may detect that data are being transmitted across the wire system 100A or wire system 100B, the wire system 200A or wire system 200B or the wire system 300A or wire system 300B, respectively.

At S722, a timer is started. For example, the controller 150, the controller 250 or the controller 350 may start a timer by noting the current time in a register address in the memory 151, the memory 251 or the memory 351, respectively. The current time may be the actual time for the location of the controller 150, the controller 250, or the controller 350, or may be an absolute time such as “0”.

At S725, the end of data usage is detected. For example, the controller 150, the controller 250 or the controller 350 may note the absence of data usage for an amount of time above a threshold, such as 15 seconds, and mark the time when the absence of data usage began in a register address in the memory 151, the memory 251 or the memory 351, respectively. At S727, a timer used to track the time of the data usage between S720 and S725 is stopped. In some embodiments, a timer is not actually stopped, and instead a feed from the timer is instead simply deactivated so that a recurrent processing algorithm which was tracking the time and the data usage no longer receives the time.

At S729, the status of usage time is updated to account for the usage between S720 and S725. For example, a count of the usage time may be obtained by the difference between the start time and the stop time, and may be added to a previous sum to update a sum of the total usage time for the corresponding system. The total usage time may be a total usage time since the system was initially assembled, or may be a total usage time since the system was last reset. For example, wire systems described herein may be reset when they are refurbished, such as when a software update is provided to the controller 150, the controller 250 or the controller 350.

At S730, a determination is made whether the end of the cycle has been reached. The end of the cycle may be determined when the corresponding system has been disconnected from both a corresponding medical device at one end and from the wall W at the other end.

If the end of the cycle has not been reached (S730=No), the method of FIG. 7 may include simply waiting for the next detection of data usage at S720 and the repeating the process from S720.

If the end of the cycle has been reached (S730=Yes), the method of FIG. 7 may return to S701 to note that the end of the current cycle has been detected and to wait for the start of a new cycle at S705.

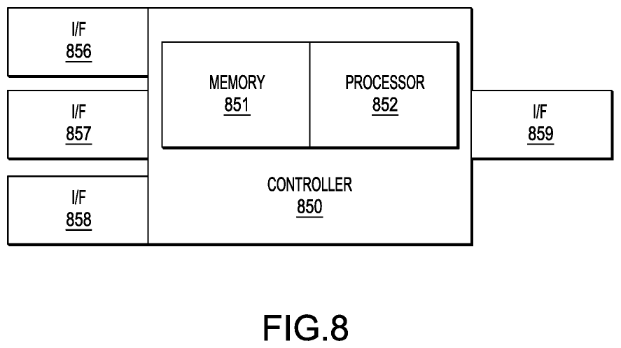

FIG. 8 illustrates a controller for wire usage monitoring, in accordance with a representative embodiment.

The controller 850 includes a memory 851 that stores instructions and a processor 852 that executes the instructions. The controller 850 also includes a first interface 856, a second interface 857, a third interface 858 and a fourth interface 859. The interfaces may interface the controller 850 to wire bodies, walls and portable electronic readers in the embodiments of FIG. 1A, FIG. 1B, FIG. 2A, FIG. 2B, FIG. 3A and FIG. 3B. For example, the first interface 856 may be an adapter configured to receive the wire body 110, the second interface 857 may be an adapter configured to be input to a sensor S in FIG. 1A, or to a port at the wall W in FIG. 1B, and the third interface 858 may be a wireless communication module including a transceiver configured to communicate with a portable electronic device that includes the user interface 681 in FIG. 6. One or more of the interfaces may include ports, disk drives, wireless antennas, or other types of receiver circuitry that connect the controller 850 to other electronic elements. One or more of the interfaces may also include user interfaces such as buttons, keys, a mouse, a microphone, a speaker, or other elements that users can use to interact with the controller 850 such as to enter instructions and receive output.

The controller 850 may perform some of the operations described herein directly and may implement other operations described herein indirectly. For example, the controller 850 may indirectly control other operations such as by generating and transmitting content to be displayed on a display of a portable electronic device. The controller 850 may directly control other operations such as logical operations performed by the processor 852 executing instructions from the memory 851 based on input received from electronic elements and/or users via the interfaces. Accordingly, the processes implemented by the controller 850 when the processor 852 executes instructions from the memory 851 may include steps not directly performed by the controller 850.

The controller 850 in FIG. 8 may correspond to any of the other controllers described herein and may include separate interfaces to electronic medical devices, to walls, and to electronic reader devices.

FIG. 9 illustrates another system for wire usage monitoring, in accordance with a representative embodiment.

In FIG. 9 the system 900 includes a controller 950 and an electronic reader device 990. The controller 950 may include a memory 951, a processor 952 and an interface 954. The interface 954 may be a wired or wireless interface to allow the electronic reader device 990 to interact with the controller 950, such as to retrieve the data in the user interface 681 shown in FIG. 6 or other types of date.

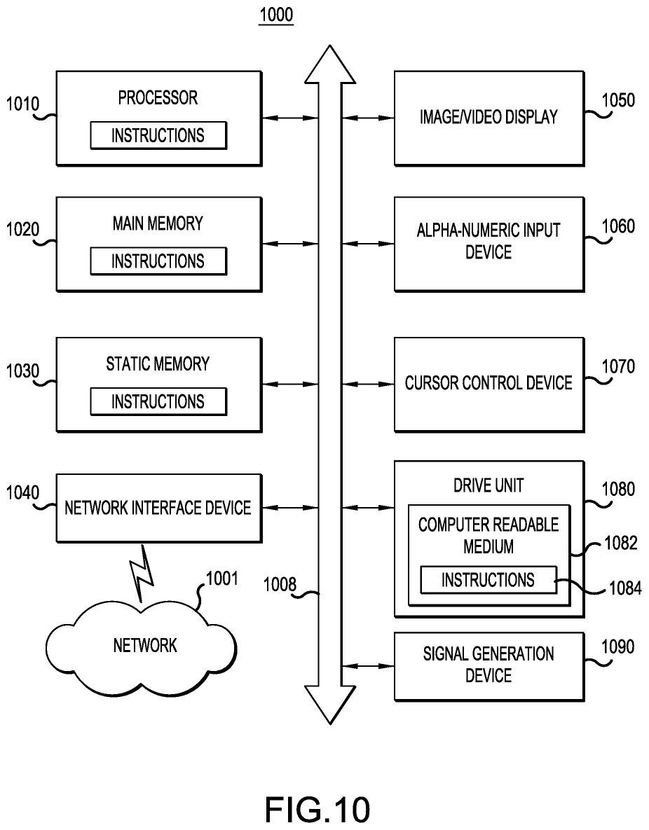

For example, the electronic reader device 990 may be dedicated only to reading controllers of wire systems as described herein. Alternatively, the electronic reader device 990 may be a general-purpose device such as a tablet or smart phone with an application installed therein to reading controller of wire systems as described herein. An example of features of an electronic reader device 990 is described below with respect to FIG. 10.

FIG. 10 illustrates a computer system, on which a method for wire usage monitoring is implemented, in accordance with another representative embodiment.

Referring to FIG. 10, the computer system 1000 includes a set of software instructions that can be executed to cause the computer system 1000 to perform any of the methods or computer-based functions disclosed herein. The computer system 1000 may operate as a standalone device or may be connected, for example, using a network 1001, to other computer systems or peripheral devices. In embodiments, a computer system 1000 performs logical processing based on digital signals received via an analog-to-digital converter.

The computer system 1000 can be implemented as or incorporated into various devices, such as a smart phone, a tablet computer, a dedicated electronic reader device, a mobile computer, a laptop computer, or any other machine capable of executing a set of software instructions (sequential or otherwise) that specify actions to be taken by that machine. The computer system 1000 can be incorporated as or in a device that in turn is in an integrated system that includes additional devices. In an embodiment, the computer system 1000 can be implemented using electronic devices that provide voice, video or data communication. Further, while the computer system 1000 is illustrated in the singular, the term “system” shall also be taken to include any collection of systems or sub-systems that individually or jointly execute a set, or multiple sets, of software instructions to perform one or more computer functions.

As illustrated in FIG. 10, the computer system 1000 includes a processor 1010. The processor 1010 may be considered a representative example of a processor of a controller and executes instructions to implement some or all aspects of methods and processes described herein. The processor 1010 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 1010 is an article of manufacture and/or a machine component. The processor 1010 is configured to execute software instructions to perform functions as described in the various embodiments herein. The processor 1010 may be a general-purpose processor or may be part of an application specific integrated circuit (ASIC). The processor 1010 may also be a microprocessor, a microcomputer, a processor chip, a controller, a microcontroller, a digital signal processor (DSP), a state machine, or a programmable logic device. The processor 1010 may also be a logical circuit, including a programmable gate array (PGA), such as a field programmable gate array (FPGA), or another type of circuit that includes discrete gate and/or transistor logic. The processor 1010 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 1000 further includes a main memory 1020 and a static memory 1030, where memories in the computer system 1000 communicate with each other and the processor 1010 via a bus 1008. Either or both of the main memory 1020 and the static memory 1030 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 1020 and the static memory 1030 are articles of manufacture and/or machine components. The main memory 1020 and the static memory 1030 are computer-readable mediums from which data and executable software instructions can be read by a computer (e.g., the processor 1010). Each of the main memory 1020 and the static memory 1030 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1000 further includes a video display unit 1050, such as a liquid crystal display (LCD), an organic light emitting diode (OLED), a flat panel display, a solid-state display, or a cathode ray tube (CRT), for example. Additionally, the computer system 1000 includes an input device 1060, such as a keyboard/virtual keyboard or touch-sensitive input screen or speech input with speech recognition, and a cursor control device 1070, such as a mouse or touch-sensitive input screen or pad. The computer system 1000 also optionally includes a disk drive unit 1080, a signal generation device 1090, such as a speaker or remote control, and/or a network interface device 1040.

In an embodiment, as depicted in FIG. 10, the disk drive unit 1080 includes a computer-readable medium 1082 in which one or more sets of software instructions 1084 (software) are embedded. The sets of software instructions 1084 are read from the computer-readable medium 1082 to be executed by the processor 1010. Further, the software instructions 1084, when executed by the processor 1010, perform one or more steps of the methods and processes as described herein. In an embodiment, the software instructions 1084 reside all or in part within the main memory 1020, the static memory 1030 and/or the processor 1010 during execution by the computer system 1000. Further, the computer-readable medium 1082 may include software instructions 1084 or receive and execute software instructions 1084 responsive to a propagated signal, so that a device connected to a network 1001 communicates voice, video or data over the network 1001. The software instructions 1084 may be transmitted or received over the network 1001 via the network interface device 1040.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, wire usage monitoring enables tracking of wire systems so that the purpose, usage and/or history of the wire systems can be stored and checked in a manner consistent with the embodiments described herein. Wire usage monitoring also enables smart functionality, such as when a wire system is connected to a specific type of sensor that is otherwise not compatible with an electronic device, so that electronic devices may be enabled to provide functions based on analysis performed by wire systems.

Although wire usage monitoring has been described with reference to several exemplary embodiments, it is understood that the words that have been used are words of description and illustration, rather than words of limitation. Changes may be made within the purview of the appended claims, as presently stated and as amended, without departing from the scope and spirit of wire usage monitoring in its aspects. Although wire usage monitoring has been described with reference to particular means, materials and embodiments, wire usage monitoring is not intended to be limited to the particulars disclosed. Rather wire usage monitoring extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72 (b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A wire system, comprising:
a wire body by which data are communicated; and
a controller dedicated to the wire body and comprising a memory that stores instructions and a processor that executes the instructions,
wherein, when executed by the processor, the instructions cause the processor to:
detect usage of the wire system when data are communicated; and
update status information for the wire system stored in the memory to track usage of the wire system.

2. The wire system of claim 1, further comprising:
an interface configured to interface the wire system to a medical monitor.

3. The wire system of claim 2, wherein the interface is configured to interface the controller to the medical monitor.

4. The wire system of claim 1, wherein, when executed by the processor, the instructions cause the controller further to:
detect information addressed to the controller;
present current settings for the wire system; and
accept updated settings for the wire system.

5. The wire system of claim 1, wherein, when executed by the processor, the instructions cause the controller further to:
detect data being communicated via the wire system;
identify a type of the data being communicated via the wire system; and
update the status information for the wire system by updating a count of usage time of the wire system.

6. The wire system of claim 1, wherein, when executed by the processor, the instructions cause the controller further to:
detect the end of a usage cycle of the wire system;
detect the start of a new usage cycle of the wire system, and
update the status information for the wire system by updating a cycle count of the wire system.

7. The wire system of claim 1, wherein, when executed by the processor, the instructions cause the controller further to:
detect a data usage of the wire system;
start a timer;
detect an end of the data usage of the wire system;
stop the timer; and
update the status information for the wire system by updating a count of usage time of the wire system.

8. The wire system of claim 1, wherein the wire body and the controller are configured to be physically integrated.

9. An operational method of a wire system, the operational method comprising a processor of the wire system executing instructions for:
detecting usage of the wire system when data are communicated via the wire system; and
updating status information for the wire system stored in a memory of the wire system to track usage of the wire system.

10. The operational method of claim 9, further comprising the processor executing instructions for:
detecting when the wire system is interfaced to a medical monitor.

11. The operational method of claim 9, further comprising the processor executing instructions for:
detecting information addressed to a controller of the wire system;
presenting current settings for the wire system; and
accepting updated settings for the wire system.

12. The operational method of claim 9, further comprising the processor executing instructions for:
detecting data being communicated via the wire system;
identifying a type of the data being communicated via the wire system; and
updating the status information for the wire system by updating a count of usage time of the wire system.

13. The operational method of claim 9, further comprising the processor executing instructions for:
detecting the end of a usage cycle of the wire system;
detecting the start of a new usage cycle of the wire system, and
updating the status information for the wire system by updating a cycle count of the wire system.

14. The operational method of claim 9, further comprising the processor executing instructions for:
detecting a data usage of the wire system;
starting a timer;
detecting an end of the data usage of the wire system;
stopping the timer; and
updating the status information for the wire system by updating a count of usage time of the wire system.

15. A tangible non-transitory computer readable storage medium that stores a computer program, wherein the computer program, when executed by a processor, causes a controller to:
detect usage of a wire system when data are communicated; and
update status information for the wire system stored in a memory of the wire system to track usage of the wire system.

16. The tangible non-transitory computer readable storage medium of claim 15, wherein the computer program, when executed by the processor further causes the computer to:
detect when the wire system is interfaced to a medical monitor.
